(19) United States
(12) Patent Application Publication
(10) Pub. No.: US 2011/0092440 A1
(43) Pub. Date: Apr. 21, 2011

(54) METHOD OF REGULATING CELL GROWTH USING A PROTEASOME INHIBITOR

(75) Inventor: Andrei Gartel, Chicago, IL (US)

(73) Assignee: THE BOARD OF TRUSTEES OF THE UNIVERSITY OF ILLINOIS, Urbana, IL (US)

(21) Appl. No.: 12/997,410

(22) PCT Filed: Jun. 12, 2009
(86) PCT No.: PCT/US09/47254

§ 371 Date: Dec. 10, 2010

Related U.S. Application Data
(60) Provisional application No. 61/060,865, filed on Jun. 12, 2008.
(60) Provisional application No. 61/167,754, filed on Apr. 8, 2009.

(51) Int. Cl. A61K 38/03; A61K 31/7105; G01N 33/53; A61P 35/00

(52) U.S. Cl. 514/18.9; 514/44 A; 435/7.21

(57) ABSTRACT

This invention provides methods and pharmaceutical compositions for regulating cell growth or inducing apoptosis in a cell, particularly a mammalian tumor cell. Specifically, the invention provides methods for inducing apoptosis in a tumor cell comprising contacting the tumor cell with a proteasome inhibitor and a thiazole antibiotic, particularly each in a suboptimal amount.

This invention relates to and claims the benefit of priority to U.S. Provisional Application Ser. Nos. 61/060,865, filed Jun. 12, 2008, and 61/167,754, filed Apr. 8, 2009. The disclosures of these two provisional applications are herein incorporated by reference in their entireties. This invention is supported by grants awarded by the National Institutes of Health under Grant Nos. 1RO1CA1294414-01A1 and 1R21CA134615-01. Thus, the United States Government has certain rights in the invention.

### BACKGROUND OF THE INVENTION

1. Field of the Invention

This application relates to the regulation of cellular growth. Specifically, the application relates to compositions, methods, and reagents useful for inducing apoptosis or inhibiting proliferation of a cell, particularly a tumor cell, by using a proteasome inhibitor and an agent that reduces the FoxM1 activity. More specifically, the agent that reduces the FoxM1 activity is a thiazole antibiotic.

2. Description of Related Art

Over the past few decades, developing effective cancer treatments has become increasingly complex and difficult. See Strausberg et al., 2004, Nature 429:469-474. One of the many difficulties arises from the lack of knowledge concerning the mechanism of cancer development, which creates problems in producing targeted therapies. The lack of target specific cancer treatments results in toxicity to not only cancer cells, but normal cells, causing severe side effects.

Generally, cancers are caused by abnormalities in the genetic material of transformed cells. Genetic abnormalities found in cancer typically affect two general classes of genes, tumor suppressors and oncogenes. An oncogene is a gene that when expressed at high levels in a cell either by genetic or epigenetic mutations activates hyperactive cell growth and protects the cell from programmed cell death (apoptosis). A proto-oncogene is a normal gene that becomes a tumor-inducing oncogene due to mutations or increased expression. One example of a proto-oncogene is the Forkhead box (Fox) M1.

FoxM1 is a transcription factor of the Forkhead family that induces the expression of genes involved in cell cycle progression and genomic stability. See Laoukili et al., 2007, Biochim Biophys Acta 1775(1):92-102. Abnormal up-regulation of FoxM1 expression is involved in the oncogenesis of basal cell carcinoma and in the majority of solid human cancers, including liver, breast, lung, prostate, uterine, colon, pancreas, and brain. See Pilarsky et al., 2004, Neoplasia 6(6):744-750; Chan, et al., 2008, J Pathol 215(3):245-252. Suppression of FoxM1 results in suppression of tumorigenesis; thus chemical compounds that target FoxM1 may act as anticancer drugs. See Radhakrishnan et al., 2006, Cancer Res 66(19):9731-9735; Adami et al., 2007, Future Oncol 3(1):1-3; Gartel, 2008, Expert Opin Ther Targets 12(6):663-665; Radhakrishnana and Gartel, 2008, Nat Rev Cancer 8(3):c1, author reply c2. Recently, thiazole antibiotics, such as Siomycin A and thiostrepton, have been shown to inhibit FoxM1 expression and induce apoptosis in human cancer cells. See Radhakrishnana et al., 2006; Bhat et al., 2008, Cell Cycle 7(12) 1851-1855. However, the exact role of FoxM1 in cancer development remains unknown.

The proteasome is a protein complex that targets ubiquitin-tagged proteins for degradation in an ATP-dependent manner in eukaryotic cells. The proteasome protein degradation pathway is involved in many cellular processes, including cell cycle regulation, apoptosis, regulation of gene expression, and responses to oxidative stress. Proteasomes have been linked to several diseases, including autoimmunity, neurodegenerative diseases, rheumatoid diseases, cancer, viral infections, and cachexia. See Dahlmann, 2007, BMC Biochem 8(Suppl 1):53. Currently, proteasome inhibitors are being used for the treatment of cancer. For example, VELCADE® (Bortezomib) was the first proteasome inhibitor approved by the U.S. Food and Drug Administration (FDA) for the treatment of multiple myeloma.

Toxicity studies indicate that VELCADE® has a very narrow therapeutic index. See Aghajanian et al., 2002, Clin Cancer Res, 8:2505-11. The recommended dose of VELCADE® is 1.3 mg/m² administered as a 3 to 5 second bolus intravenous injection, and dose adjustment must be considered to manage adverse events that occur during treatment. Adverse reactions associated with VELCADE® include asthenic conditions, diarrhea, nausea, constipation, peripheral neuropathy, vomiting, pyrexia, thrombocytopenia, psychiatric disorders, change in appetite, neutropenia, neuralgia, leucopenia, and anemia. See FDA web site: http://www.accessdata.fda.gov/drugsatfda_docs/label/2008/021602s0151bl.pdf; and Aghajanian et al., 2002. The narrow therapeutic index of VELCADE®, coupled with the various side effects associated with the drug, suggest that improvement is needed for a better proteasome inhibitor or a better therapeutic regimen using proteasome inhibitors for the treatment of cancer.

Therefore, much improvement is needed for a better proteasome inhibitor for the treatment proteasome-related diseases, including cancer. Further, a better therapeutic regimen using proteasome inhibitors for the treatment of cancer is needed.

### SUMMARY OF THE INVENTION

This invention provides methods and pharmaceutical compositions for regulating cell growth or inducing apoptosis in a cell, particularly a mammalian cell, more particularly a mammalian tumor cell. Specifically, the invention provides methods for inducing apoptosis in a tumor cell comprising contacting the tumor cell with a proteasome inhibitor and a thiazole antibiotic.

In one aspect, the invention provides methods for inducing apoptosis in a tumor cell by contacting the tumor cell with a proteasome inhibitor and a thiazole antibiotic, wherein the combination of proteasome inhibitor and thiazole antibiotic is effective in inducing apoptosis in the tumor cell. In certain embodiments of this aspect, the proteasome inhibitor is MG132, MG115, VELCADE®, lactacystin, or PSI. In certain particular embodiments, the proteasome inhibitor is VELCADE®. In certain embodiments, the thiazole antibiotic is Siomycin A, thiostrepton, sporangiomycin, nosiheptide, multhiomycin, micrococcin or thiocillin. In certain particular embodiments, the thiazole antibiotic is Siomycin A or thiostrepton. In further embodiments, the proteasome inhibitor is VELCADE® and the thiazole antibiotic is Siomycin A or thiostrepton.

In accordance with this aspect, the invention provides certain embodiments wherein the tumor cell is contacted with a suboptimal amount of proteasome inhibitor and a suboptimal amount of thiazole antibiotic. As provided herein, said suboptimal amounts are advantageous because they are associated with reduced incidence or severity or both of adverse or otherwise undesirable side-effects produced by administration of the proteasome inhibitor or thiazole antibiotic in optimal amounts, while retaining therapeutic efficacy when administered in combination. In certain embodiments, suboptimal amount of a proteasome inhibitor is from about 2 μg/kg to about 400 μg/kg. In additional embodiments, the suboptimal amount of a thiazole antibiotic is from about 800 μg/kg to about 5 mg/kg. In certain particular embodiments, the proteasome inhibitor is VELCADE® at a suboptimal amount of about 2 μg/kg and the thiazole antibiotic is thiostrepton at a suboptimal amount of about 1.3 mg/kg. In certain other particular embodiments, the suboptimal amount of VELCADE® is about 4 μg/kg, and the suboptimal amount of thiostrepton is about 1.6 mg/kg.

In another aspect, the invention provides pharmaceutical compositions for inducing apoptosis in a tumor cell, comprising a proteasome inhibitor as described herein and a thiazole antibiotic as described herein, and at least one excipient, diluent, or carrier, wherein the combination of a proteasome inhibitor and a thiazole antibiotic is effective in inducing apoptosis in the tumor cell. In certain particular embodiments of this aspect, the pharmaceutical compositions comprise a proteasome inhibitor VELCADE®. In other particular embodiments, the pharmaceutical compositions comprise a thiazole antibiotic Siomycin A or thiostrepton. In further particular embodiments, the pharmaceutical composition comprises VELCADE® and thiostrepton.

In particular embodiments, the pharmaceutical compositions comprise a suboptimal amount of the proteasome inhibitor and a suboptimal amount of the thiazole antibiotic, wherein the combination of a proteasome inhibitor and a thiazole antibiotic in suboptimal amounts is sufficient to induce apoptosis in the tumor cell. In certain embodiments, the suboptimal amount for the proteasome inhibitor is from about 2 μg/kg to about 400 μg/kg, particularly from about 2 μg/kg to about 40 μg/kg, and more particularly from about 2 μg/kg to about 30 μg/kg, and from about 2 μg/kg to about 20 μg/kg. In other embodiments, the suboptimal amount for the thiazole antibiotic is from about 800 μg/kg to about 5 mg/kg. In certain particular embodiments, the proteasome inhibitor is VELCADE® at a suboptimal amount of about 2 μg/kg and the thiazole antibiotic is thiostrepton at a suboptimal amount of about 1.3 mg/kg. In certain other particular embodiments, the suboptimal amount of VELCADE® is about 4 μg/kg, and the suboptimal amount of thiostrepton is about 1.6 mg/kg.

In yet a further aspect, the invention provides methods for inducing apoptosis in a tumor cell that expresses FoxM1 protein, comprising the step of contacting the tumor cell with a proteasome inhibitor and at least one agent that reduces FoxM1 activity. In certain embodiments, suitable agents that reduce FoxM1 activity include without limitation a thiazole antibiotic, a FoxM1 siRNA, and a p19ARF peptide. In certain particular embodiments, the agent is a thiazole antibiotic. In other particular embodiments, the thiazole antibiotic is Siomycin A or thiostrepton. In further embodiments, the proteasome inhibitor is selected from MG132, MG115, VELCADE®, lactacystin, or PSI. In particular embodiments, the proteasome inhibitor is VELCADE®.

In accordance with this aspect of the invention, pharmaceutical compositions comprising a proteasome inhibitor as described herein and an agent that reduces FoxM1 activity, and at least one excipient, diluent or carrier are also provided, wherein the combination of the proteasome inhibitor and the agent that reduces FoxM1 activity is effective in inducing apoptosis in the tumor cell.

In a further aspect, the invention provides methods for inhibiting FoxM1 activity in a tumor cell comprising the step of contacting the cell with a proteasome inhibitor. In certain embodiments, the proteasome inhibitor is MG132, MG115, VELCADE®, lactacystin, or PSI. In certain particular embodiments, the proteasome inhibitor is VELCADE®. In certain other embodiments, the invention provides methods for inhibiting FoxM1 activity in a tumor cell by contacting the cell with a proteasome inhibitor and a thiazole antibiotic. In other embodiments, the thiazole antibiotic is Siomycin A, thiostrepton, sporangiomycin, nosiheptide, multhiomycin, micrococcin or thiocillin. In certain particular embodiments, the thiazole antibiotic is Siomycin A or thiostrepton. In certain particular embodiments, the proteasome inhibitor is VELCADE® and the thiazole antibiotic is thiostrepton.

In yet another aspect, the present invention provides methods for inhibiting proteasome activity in a cell comprising the step of contacting the cell with a thiazole antibiotic. In certain embodiments, the thiazole antibiotic is Siomycin A, thiostrepton, sporangiomycin, nosiheptide, multhiomycin, micrococcin or thiocillin. In certain other embodiments, the methods for inhibiting proteasome activity in a cell comprising the step of contacting the cell with a thiazole antibiotic and a proteasome inhibitor. Advantageously, when a combination of a thiazole antibiotic and a proteasome inhibitor is used, each compound can be used at a suboptimal amount in the combination. As provided herein, said suboptimal amounts are advantageous because they are associated with reduced incidence or severity or both of undesirable side-effects produced by administration of the thiazole antibiotic and/or proteasome inhibitor in optimal amount, while retaining therapeutic efficacy when administered in combination. In addition, applying suboptimal amounts of each of the thiazole antibiotic and proteasome inhibitor in a combination allows an ordinarily skilled clinician to titrate and adapt doses that retain drug efficacy and yet avoid the side effects.

In a further aspect, this invention provides methods for identifying a compound having proteasome inhibitory activity in a cell by determining the reduction of FoxM1 activity in the cell by the compound, wherein the cell expresses FoxM1, the method comprising the steps of contacting the cell with the compound, and assaying for FoxM1 activity in the cell. In certain particular embodiments, the compound is a thiazole antibiotic. In yet another aspect, the invention provides methods for identifying a thiazole antibiotic that inhibits proteasome activity in a cell, comprising the steps of contacting the cell with said thiazole antibiotic and detecting reduced proteasome activity in the cell.

Specific embodiments of the present invention will become evident from the following more detailed description of certain preferred embodiments and the claims.

### BRIEF DESCRIPTION OF THE FIGURES

FIG. 1A shows photographs of immunoblot analysis of Hdm2, p53, Mcl-2 and p21 protein levels in U2OS cells treated with Siomycin A or MG132. The protein levels of β-actin were used as a control. FIG. 1B depicts a bar graph of TNF-α-induced NF-κB-dependent luciferase activity in experiments where the luciferase activity was mediated by the NF-κB responsive elements. The results shown were mean±SD of three independent experiments. FIG. 1C shows a bar graph of the effects of thiazole antibiotics and proteasome inhibitors on proteasome activity. The results shown were mean±SD of three independent experiments.

FIG. 2 depicts the effects of proteasome inhibitors on FoxM1 transcriptional activity and FoxM1 and caspase 3 expressions. FIGS. 2A(1) and (2) show bar graphs indicating doxycycline-induced FoxM1-dependent luciferase activity in U2OS-C3-Luc cells treated with proteasome inhibitors (“Nor” in FIG. 2A(2) indicates MG115). The results shown in FIG. 2A(1) were mean±SD of three independent experiments. FIG. 2B depicts photographs of immunoblot analysis of FoxM1 and the active form of caspase 3 protein levels in different tumor cells treated with proteasome inhibitors. FIGS. 2C and 2D show photographs of immunoblot analysis of endogenous and exogenous FoxM1 protein levels in cells treated with proteasome inhibitors or thiazole antibiotics. The protein levels of β-actin were used as a control.

FIG. 3 shows results of fluorescence-activated cell sorting (FACS) analysis detecting Annexin V-PE/7AAD staining in cells treated with proteasome inhibitors. The numbers in the parentheses indicate the percentage of cells undergoing apoptosis. X-axis: Annexin V log intensity; y-axis: PE/7AAD log intensity.

FIG. 4 depicts photographs of immunoblot analysis of the active (cleaved) capase 3 in U2OS-C3 cells induced with doxycycline and treated with either the proteasome inhibitor VELCADE® or a non-proteasome inhibitor anti-cancer drug doxorubicin. The protein levels of β-actin were used as a control.

FIG. 5 depicts photographs of immunoblot analysis showing synergistic effects of thiazole antibiotic Siomycin A with proteasome inhibitor MG132 on apoptosis in different tumor cells: U205, osteosarcoma cells; BxPC3, human pancreatic cancer cells; and CEM, lymphoblastic leukemia cells.

FIG. 6 shows photographs of immunoblot analysis of different types of tumor cells treated with Siomycin A (Sio) and VELCADE® (Vel) at indicated concentrations. FIGS. 6A-6C, Mia Paca, BxPC3, HPAC: human pancreatic cancer cells; FIG. 6D, MDAMB231: human breast cancer cells.

FIG. 7 depicts photographs of immunoblot analysis showing synergistic effects of thiazole antibiotic thiostrepton with proteasome inhibitor MG132 on apoptosis in different tumor cells: U2OS-C3, osteosarcoma cells (un-induced by doxycycline); and HL60, leukemia cells.

FIG. 8 A depicts photographs of immunoblot analysis showing synergistic effects of thiazole antibiotic thiostrepton with proteasome inhibitor VELCADE® on apoptosis in two prostate cell lines DU145 and PC-3. FIG. 8 B depicts a graph showing synergistic effects of thiostrepton and VELCADE® on the reduction of cell viability (x-axis, concentrations of thiostrepton; y-axis, percentage of viable cells). FIG. 8 C shows a Combination Index-Fractional Effect plot depicting the effects of different concentrations of thiostrepton and VELCADE®, alone or in combination, on cell survival. A combination index (CI) value less than 1 indicated synergy, a CI value of 1 would indicate additive effects and a CI value larger than 1 would indicate antagonistic effects.

FIG. 9 depicts results of FACS analysis detecting Annexin V-PE/7AAD staining in cells treated with thiazole antibiotic thiostrepton (Thio) and proteasome inhibitor VELCADE® (Vel), separately or in combination. The numbers below each panel indicate the percentage of cells undergoing apoptosis. X-axis: Annexin V log intensity; y-axis: PE/7AAD log intensity.

### DETAILED DESCRIPTION OF THE INVENTION

This invention provides methods and reagents for inhibiting proliferation or inducing apoptosis in a cell, particularly a mammalian cell, and more particularly a mammalian tumor cell. Specifically, the invention provides methods and reagents for inducing apoptosis in a tumor cell using a combination of a proteasome inhibitor and a thiazole antibiotic. Further, the invention provides methods for inhibiting proteasome activity using a thiazole antibiotic.

As used herein, the term “thiazole,” “thiazole compound,” “thiazole antibiotic” or “thiazole antibiotic compound” refers to a thiazole compound that negatively affects proteasome activity. In particular embodiments, the thiazole or thiazole compound is a thiazole antibiotic. Thiazole antibiotics are known to interact with the bacterial 23S ribosomal RNA thereby inhibiting bacterial protein translation. This class of compounds, however, is not known to block eukaryotic protein synthesis. See Bhat et al., 2009, PLoS One 4:e5592. Suitable thiazoles or thiazole antibiotics for use in the present invention include without limitation Siomycin A, thiostrepton, sporangiomycin, nosiheptide, multhiomycin, micrococcin or thiocillin. See Prange et al., 1977, Nature 265:189-190; Cundliffe et al., 1975, Antimicrob Agents Chemother. 8:1-4; Endo et al., 1978, J. Antibiotics 31:623-625; and Brown et al., 2009, PNAS, USA 106:2549-2553. In certain particular embodiments, the thiazole compound for use in the current invention is a thiazole antibiotic Siomycin A or thiostrepton.

It has been described in a co-owned co-pending U.S. patent application Ser. No. 11/865,410 (filed Oct. 1, 2007, published as U.S. Patent Application Publication No. U.S. 2008/0152618) that thiazole antibiotics inhibited FoxM1 transcriptional activity as well as FoxM1 mRNA and protein expression. FoxM1 as a transcriptional factor positively auto-regulates its own gene expression. The disclosures of U.S. Patent Application Publication No. 2008/0152618 and all other references cited throughout the application are hereby incorporated by reference in their entireties.

FoxM1 is one of the most over-expressed genes in human solid tumors. Over-expression of FoxM1 protein has been observed in hepatocellular carcinomas, pancreatic carcinomas, breast cancers, non-small cell lung carcinomas, anaplastic astrocytomas, glioblastomas, prostate cancer, colon cancer, uterine cancer, basal cell carcinomas, and intrahepatic cholangiocarcinomas. See Pilarsky et al., 2004, Neoplasia 6(6):744-750; Chan et al., 2008, J Pathol 215(3):245-252; and Bhat et al., 2009. It has been reported that thiazole antibiotics decreased FoxM1 expression, increased the expression of the apoptosis indicator caspase 3 and induced apoptosis. See U.S. Patent Publication No. 2008/0152618 Inhibition of FoxM1 expression and induction of apoptosis by thiazole antibiotics suggested the potentials of these thiazole compounds in cancer therapy.

As described herein, it was unexpectedly discovered in the instant application that thiazole antibiotics as described herein inhibited proteasome activity. Methods for determining proteasome activities are known in the art and further described in the instant application. Commercially available kits for proteasome activity analysis include without limitation the 20S Proteasome Activity Assay Kit (Millipore, Billerica, Mass.).

Proteasomes have been implicated in the development of various diseases, including without limitation autoimmune/rheumatoid diseases, neurodegenerative disease, cancer, cardiac dysfunction, cataract formation, viral infections, and cachexia. See Dahlmann, 2007 Inhibition of proteasome activity has both pro- and anti-apoptotic effects. The anti-apoptotic proteasomal effects were predominantly found in neoplastic cells wherein pro-apoptotic proteins such as p53 were degraded by proteasomes. In addition, neovascularization, cell adhesion and intravasation, processes often required in fast growing tumors are proteasome-dependent. Thus, a main treatment strategy for treating these types of neoplasias involves the induction of apoptosis through the introduction of proteasome inhibitors such as bortezomib (VELCADE®) and NPI-0052 into the neoplastic cells.

Pro-apoptotic proteasomal activity was observed in umbilical vein endothelial cells, primary thymocytes and neurons, wherein anti-apoptotic proteins such as Bc12 were degraded by proteasomes. Thus, therapeutic use of proteasome inhibitors can prevent or treat disease conditions such as certain neural degeneration caused by neuronal cell death. Additionally, overactive proteasomes may lead to accelerated protein degradation, which is a hallmark of many pathological conditions such as chronic kidney disease, type I diabetes mellitus, cancer cachexia and sepsis or burn-injury induced muscle atrophy. It has been shown that proteasome inhibitors suppressed muscle atrophy induced by sepsis or burn injuries. See Fang et al., 1998, Clin. Sci. 95:225-233; and Hobler et al., 1998 Am J. Physiol. 274:R30-R37. In addition, proteasome inhibitors can be useful as immunosuppressive agents in autoimmune disease by modulating antigen processing and MHC class I-restricted antigen presentation. Proteasome inhibitors can also be useful for controlling activated proteasome-mediated neurological inflammation caused by retroviral infection. See Groettrup et al., 1999 DDT 4:63-71; and Ott et al. 2003 J. Viol. 77:3384-3393. Using thiazole antibiotics to inhibit proteasome activity, however, has not been previously reported.

As described herein, thiazole antibiotics not only inhibited FoxM1 expression but also inhibited proteasome activity. The inhibition of proteasome activity by thiazole antibiotics was coincidental with increased protein levels of a series of genes involved in cell cycle regulation, such as p21, Mcl-1, p53 and Hdm2, in a manner similar to known proteasome inhibitors. It was further unexpectedly discovered in the instant application that cells treated with previously known proteasome inhibitors also exhibited reduced expression of FoxM1 gene. The negative effects of proteasome inhibitors on FoxM1 expression and the inhibitory effects of thiazole antibiotics on proteasome activity have not been previously reported.

Thus, in a further aspect, this invention provides methods for identifying a compound that has proteasome inhibitory activity in a cell by determining the reduction of FoxM1 activity in the cell by the compound, wherein the cell expresses FoxM1, the method comprising the steps of contacting the cell with the compound, and assaying for FoxM1 activity in the cell. In certain particular embodiments, the compound is a thiazole antibiotic. In yet another aspect, this invention provides methods for identifying a thiazole antibiotic that inhibits proteasome activity in a cell, comprising the steps of contacting the cell with said thiazole antibiotic and detecting reduced proteasome activity in the cell.

Attempts have been made to apply proteasome inhibitors to cancer therapies. Bortezomib (VELCADE®) was the first proteasome inhibitor approved by the U.S. Food and Drug Administration for the treatment of multiple myeloma. Cancer therapy using VELCADE®, however, is hindered by the narrow therapeutic index of the drug as a result of high toxicity. See Aghajanian et al., 2002, Clin Cancer Res, 8(8):2505-11. It was surprisingly discovered in the instant application that a thiazole antibiotic together with a proteasome inhibitor synergistically induced apoptosis in a tumor cell. Comparable or even higher levels of tumor cell apoptosis can be achieved using less amount of proteasome inhibitor when combined with a thiazole antibiotic. Similarly, it was observed in the instant application that less amount of a thiazole antibiotic was required to achieve comparable or even higher levels of apoptosis when the tumor cells were treated with a thiazole antibiotic in combination with a proteasome inhibitor.

Thus, in certain advantageous embodiments of this aspect, the invention provides methods for inducing apoptosis in a tumor cell comprising the step of contacting the tumor cell with a suboptimal amount of a proteasome inhibitor and a suboptimal amount of a thiazole antibiotic, wherein the combination of proteasome inhibitor and thiazole antibiotic both present in the suboptimal amounts is effective in inducing apoptosis in the tumor cell. In certain particular embodiments, the proteasome inhibitor is VELCADE® and the thiazole antibiotic is thiostrepton.

As used herein, the term “suboptimal amount” refers to a dosage amount of a therapeutic compound that is less than the clinically approved therapeutically effective amount when the compound is used alone. In certain advantageous embodiments, both the proteasome inhibitor and the thiazole antibiotic are administered in suboptimal amounts to achieve synergistic effects of apoptosis induction without the side effects such as, inter alia, non-tumor cell cytotoxicity.

In certain embodiments, the suboptimal amount for the proteasome inhibitor is from about 2 μg/kg to about 400 μg/kg, particularly from about 2 μg/kg to about 40 μg/kg, and more particularly from about 2 μg/kg to about 30 μg/kg, and from about 2 μg/kg to about 20 μg/kg. In other embodiments, the suboptimal amount for the thiazole antibiotic is from about 800 μg/kg to about 5 mg/kg, more particularly from about 1 mg/kg to about 4 mg/kg. In certain particular embodiments, the proteasome inhibitor is VELCADE® at a suboptimal amount of about 2 μg/kg and the thiazole antibiotic is thiostrepton at a suboptimal amount of about 1.3 mg/kg. In certain other particular embodiments, the suboptimal amount of VELCADE® is about 4 μg/kg, and the suboptimal amount of thiostrepton is about 1.6 mg/kg. These amounts were calculated based on the results obtained from the in vitro assays described herein using thiostrepton and VELCADE® as examples, the results obtained from which would provide guidelines for in vivo treatment. The information regarding conversion dosage form between mg/kg and mg/m² is well known in the art. See for example, Friereich et al., 1966, Cancer Chemother. Rep. 50:219-244; and hftp://web.ncifcrfgov/rtp/LASP/intra/acuc/fred/guidelines/ACUC42EquivSurfAreaD osageConversion.pdf. One of ordinarily skilled clinician would be able to titrate and modify the amounts described herein for developing effective therapeutic regimens.

As used herein, the term “proteasome inhibitor” refers to a non-thiazole antibiotic compound that inhibits proteasome activity. Suitable proteasome inhibitors include without limitation MG132 (Z-L-leucyl-L-leucyl-L-leucinal), MG115 (Z-L-leucyl-L-leucyl-L-norvalinal), VELCADE® (bortezomib, pyrazylcarbony-phenylalanyl-leucyl-boronate, Millennium Pharmaceuticals, Cambridge, Mass.), lactacystin, PSI (N-benzyloxycarbony-Ile-Glu-(O-t-butyl)-Ala-leucinal) (SEQ ID NO:9), NPI-0052 (Salinsporamide-A), and ALLN (Acetyl-L-Leucyl-L-Leucyl-L-Norleucinal). In certain particular embodiments, the proteasome inhibitor is MG132, MG115, VELCADE®, lactacystin or PSI. In certain other particular embodiments, the proteasome inhibitor is VELCADE®.

In another aspect, the invention provides methods for inhibiting proteasome activity in a cell, comprising the step of contacting the cell with a thiazole antibiotic, and optionally a proteasome inhibitor. In accordance with this aspect, the inventive methods can lead to alleviation of proteasome-mediated disease conditions. One of skill in the art can examine the cause of a disease condition and determine whether applying the method for inhibiting proteasome activity using the thiazole antibiotic as described herein will benefit the disease condition. In certain embodiments of this aspect, the methods for inhibiting proteasome activity in a cell comprise the step of contacting the cell with a thiazole antibiotic in combination with a proteasome inhibitor.

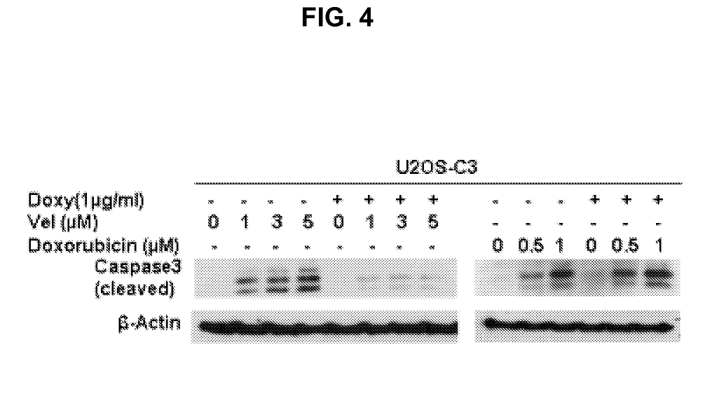

It was described in the instant application that U2OS-C3 cells over-expressing FoxM1 induced by doxycycline exhibited reduced levels of caspase 3 protein as compared to un-induced U2OS-C3 cells upon the treatment of VELCADE® (see FIG. 4). This observation may explain the ineffectiveness of using a proteasome inhibitor alone in inhibiting the proliferation or inducing apoptosis in FoxM1-overexpressing cancer cells. Thus, in a further aspect, the invention provides methods for inducing apoptosis in a tumor cell that expresses FoxM1 protein, comprising the step of contacting the tumor cell with a proteasome inhibitor and at least one agent that reduces FoxM1 activity. In certain advantageous embodiments, the agent that reduces FoxM1 activity is a thiazole antibiotic as described herein.

As used herein the term “agent” refers to a therapeutic molecule or therapeutic compound that is not a proteasome inhibitor as defined herein and that the therapeutic molecule or therapeutic compound reduces FoxM1 activity. In certain particular embodiments, the agent that reduces FoxM1 activity is a thiazole antibiotic.

Efforts have been made to discover and develop agents that reduce the activity of FoxM1 in FoxM1-expressing tumor cells. It has been shown that tumor suppressor p19-ARF, pRb, p16 or p53 inhibited FoxM1 activity. Additionally, small interfering RNAs (siRNAs) have been used to knock down FoxM1 activity in tumor cells, and peptides comprising p19ARF amino acid residues 26-44 have been shown to inhibit FoxM1 nuclear localization and transcription activity (see co-owned, co-pending U.S. patent applications Ser. Nos. 10/809144, 11/150756 and 11/571,030, published as U.S. Patent Application Publication Nos. 2005/0032692, 2006/0014688, and 2009/0075376, respectively; see also Kalin et al., 2006, Cancer Res. 66:1712-1720; Kim et al., 2006, Cancer Res. 66:2153-2161; Kalinichenko et al., 2004, Genes Dev. 18:830-850; and Gusarova et al., 2007, J. Clin. Invest. 117:99-111). Exemplary siRNAs effective in down-regulating FoxM1 gene transcription include without limitation 5′-caa cag gag ucu aau caa g uu-3′(SEQ ID NO:1), 5′-gga cca cuu ucc cua cuu u uu-3′(SEQ ID NO:2), 5′-gua gug ggc cca aca aau u uu-3′(SEQ ID NO:3), 5′-gcu ggg auc aag auu auu a uu-3′(SEQ ID NO:4). Exemplary p19 ARF peptides that are effective in inhibiting FoxM1 activity include without limitation (D-Arg)₉-KFVRSRRPRTASCALAFVN (SEQ ID NO:5), KFVRSRRPRTASCALAFVN (SEQ ID NO:6), and KFVRSRRPRTASCALAFVNMLLRLERILRR (SEQ ID NO:7).

In another aspect, the invention provides a proteasome inhibitor and a thiazole antibiotic for use in therapies in treating cancer or inducing apoptosis in a cancer cell, including without limitation, multiple myelomas, osteosarcomas, leukemias, hepatocellular carcinomas, pancreatic carcinomas, breast cancers, non-small cell lung carcinomas, anaplastic astrocytomas, glioblastomas, prostate cancer, colon cancer, uterine cancer, basal cell carcinomas, and intrahepatic cholangiocarcinomas. In particular embodiments, the cancer is multiple myeloma, osteosarcoma, or leukemia. In another aspect, the invention provides the use of a proteasome inhibitor and a thiazole antibiotic in the manufacture of medicaments for the treatment of cancer.

The invention also provides pharmaceutical compositions for inducing apoptosis in a tumor cell, comprising a proteasome inhibitor and a thiazole antibiotic, and at least one excipient, diluent or carrier, wherein the combination of proteasome inhibitor and thiazole antibiotic is effective in inducing apoptosis in the tumor cell. In a further aspect, the invention provides pharmaceutical compositions comprising a proteasome inhibitor and at least one agent that reduces FoxM1 activity.

The pharmaceutical compositions of the invention may contain formulation materials such as pharmaceutically acceptable carriers, diluents, excipients for modifying, maintaining, or preserving, in a manner that does not hinder the activities of the therapeutic compounds or molecules described herein, for example, pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; Triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

The primary vehicle or carrier in a pharmaceutical composition may be either aqueous or non-aqueous in nature. For example, a suitable vehicle or carrier for injection may be physiological saline solution, or artificial cerebrospinal fluid. Optimal pharmaceutical compositions can be determined by a skilled artisan depending upon, for example, the intended route of administration, delivery format, desired dosage and recipient tissue. See, e.g., REMINGTON'S PHARMACEUTICAL SCIENCES, supra. Such compositions may influence the physical state, stability, and effectiveness of the composition.

The pharmaceutical composition to be used for in vivo administration typically is sterile and pyrogen-free. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect.

The dosing frequency will depend upon the pharmacokinetic parameters of a proteasome inhibitor and a thiazole antibiotic in the formulation. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, subcutaneous, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

Pharmaceutical compositions of the invention can be administered alone or in combination with other therapeutic agents, in particular, in combination with other cancer therapy agents. Such agents generally include radiation therapy or chemotherapy. Chemotherapy, for example, can involve treatment with one or more of the following agents: anthracyclines, taxol, tamoxifene, doxorubicin, 5-fluorouracil, nucleoside analogs, and other drugs known to one skilled in the art. In patient with non-cancer angiogenesis-dependent diseases, pharmaceutical compositions of the invention can be administered alone or in combination with other therapeutic agents, for example, agents for treating inflammatory disorders such as rheumatoid arthritis or psoriasis, and agents for treating disorders associated with inappropriate invasion of vessels.

In one embodiment, the methods of the invention can be advantageously performed after surgery where solid tumors have been removed as a prophylaxis against metastases.

The pharmaceutical compositions of the invention can be administered to a patient in need thereof. The term “patient” as used herein refers to an animal, especially a mammal. In certain particular embodiments, the mammal is a human with cancer.

The Examples, which follow, are illustrative of specific embodiments of the invention, and various uses thereof. They are set forth for explanatory purposes only, and are not to be taken as limiting the invention.

### EXAMPLES

### Experimental Procedure

### 1. Cell Lines, Media and Chemical Compounds

U266 and RPMI8226 multiple myeloma cell lines, and HL-60 leukemia cell line were purchased from American Type Culture Collection (Manassas, Va.) and were grown in RPMI1640 medium (Invitrogen, Carlsbad, Calif). The following cell lines were grown in DMEM medium (Invitrogen): BxPC3, CEM, DU145, Mia Paca, HPAC, MDAMB231, PC3, U2OS osteosarcoma cells, U2OS-C3 cells, a U2OS-derived cell line stably expressing doxycycline-inducible FoxM1-GFP fusion protein (Kalinichenko et al. 2004); U2OS-C3-Luc cells, a U2OS-C3 derived cell line stably expressing, in addition to the doxycycline-inducible FoxM1-GFP, the firefly luciferase under the control of multiple FoxM1 responsive elements (Radhakrishnan et al. 2006); and 293T-NF-κB-Luc cell line that was stably transfected with an NF-κB-Luc reporter construct. In all cases the media were supplemented with 10% fetal bovine serum (Atlanta Biologicals, Lawrenceville, Ga.) and 1% penicillin-streptomycin (Invitrogen) and the cell lines were kept at 37° C. in 5% CO₂. Siomycin A (NCI, Bethesda, Md.; or Tebu-Bio, Boechout, Belgium, Catolog No. 170BIA-S1136-1), thiostrepton (Sigma, St. Louis, Mo.), doxorubicin (Sigma), MG115 (Sigma), MG132 (Calbiochem, San Diego, Calif.) and VELCADE® (Millenium Pharmaceuticals) were dissolved in dimethylsulfoxide (DMSO), doxycycline (Clontech, Mountain View, Calif.) was dissolved in phosphate-buffered saline (PBS) and TNF-α (R&D Systems, Minneapolis, Minn.) was dissolved in PBS containing 0.1% bovine serum albumin.

### 2. Immunoblot Analysis

Cancer cells of different origin treated as indicated were harvested and lysed using the IP buffer (20mM HEPES, 1% Triton X-100, 150 mM NaCl, 1 mM EDTA, 1 mM EGTA, 100 mM NaF, 10 mM Na₄P₂O₇, 1 mM sodium othrovanadate, 0.2 mM PMSF supplemented with protease inhibitor tablet (Roche Applied Sciences, Indianapolis, IN)). Protein concentration was determined by using the Bio-Rad Protein Assay (BIO-RAD, Hercules, Calif.). Isolated proteins were separated on 8% or 10% SDS-PAGE and transferred to PVDF membrane (Millipore, Billerica, Mass.). Immunoblot analysis was carried out as described in Radhakrishnan et al., 2004, and Radhakrishnan and Gartel 2006 with antibodies specific for FoxM1 (a gift from Dr. Robert Costa's lab at the University of Illinois at Chicago), cleaved caspase-3 (Cell Signaling, Beverly, Mass.), p53 (Santa-Cruz Biotechnology, Santa Cruz, Calif.), p21 (BD-Pharmingen, San Jose, Calif.), Hdm2 (Santa-Cruz Biotechnology), Mcl-1 (Lab Vision, Fremont, Calif.), and β-actin (Sigma).

### Example 1

### Thiazole Antibiotics Inhibited Proteasome Activities

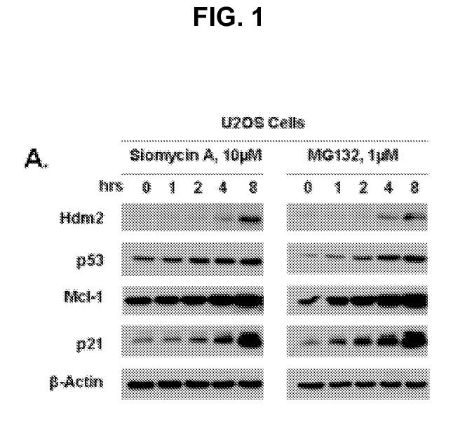

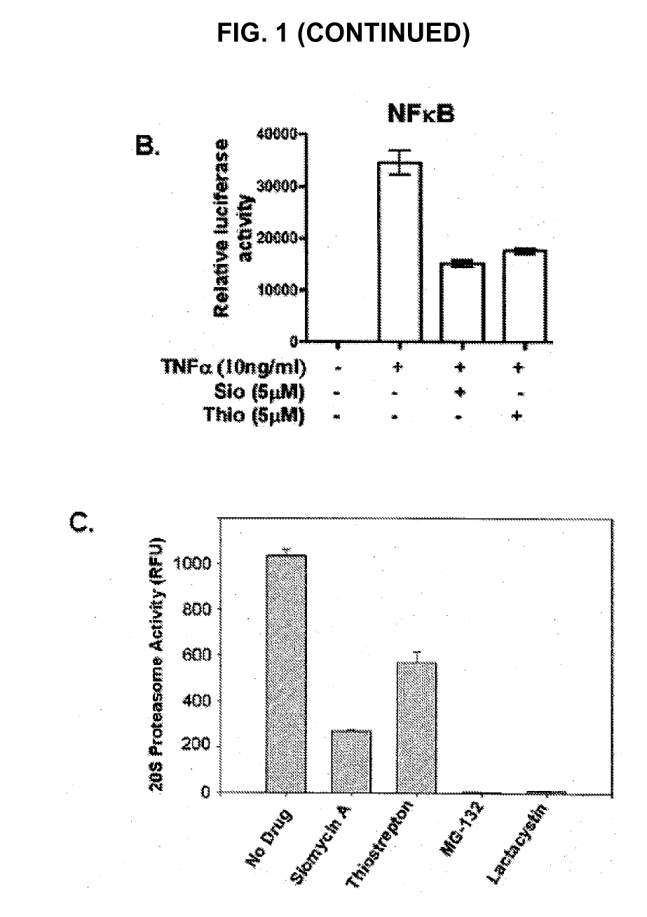

It has been reported previously that thiazole antibiotics such as Siomycin A and thiostrepton inhibited FoxM1 transcriptional activity as well as FoxM1 protein expression in cells (see U.S. Patent Application Publication No. 2008/0152618; see also Radhakrishnan et al., 2006, Cancer Res 66:9731-35; Bhat et al., 2009; and Halasi et al., 2009, Cell Cycle 8: 1966-1967). It was unexpectedly discovered that, in contrary to FoxM1, treatment of Siomycin A resulted in an increase or stabilization of several other cellular proteins, such as, p21, Mcl-1, p53 and Hdm2 (FIG. 1A).

Proteasome inhibitors have been shown to increase the protein levels of p21, Mcl-1, p53 and Hdm2 in the cells (Nencioni et al., 2007, Leukemia 21:30-6; Matta et al., 2005, Cancer Biol Ther 4:77-82). The effects of Siomycin A on the protein levels of these genes in the cells were compared with those of a proteasome inhibitor MG132. As shown in FIG. 1A, Siomycin A increased the cellular protein levels of Hdm2, p53, Mcl-1 and p21 in a manner similar to the proteasome inhibitor MG132, while the levels of beta-actin protein were not affected by the treatment of Siomycin A.

Proteasome inhibitors were known to inhibit the activity of NF-κB via the stabilization of its negative regulator IκB-α (Nakanishi et al., 2005, Nat Rev Cancer 5:297-309; Nencioni et al., 2007). The effects of Siomycin A and thiostrepton on NF-κB-dependent transcriptional activity were tested. 293T cells stably transfected with an NF-κB-Luc reporter construct (293T-NF-κB-Luc) were induced with 10 ng/mL TNF-αfor 24 hours. The next day, the cells were treated with Siomycin A or thiostrepton for an additional 10 hours, and followed by luciferase assay. The luciferase activity was determined by using the Luciferase Assay System (Promega, Madison, Wis.) according to the manufacturer's instructions. The data were normalized based on the amount of proteins in the samples. The results as shown in FIG. 1B indicated that both thiostrepton and Siomycin A suppressed TNF-α-induced NF-κB transcriptional activity, similar to proteasome inhibitors (see Sors et al., 2006, Blood 107:2354-63).

The effects of Siomycin A and thiostrepton on the 20S proteasome were compared with proteasome inhibitors MG132 and lactacystin using the Proteasome Activity Assay Kit following the manufacturer's instructions (Millipore). The assay principle was based on the detection of free fluorophore-labeled 7-Amino-4-methylcoumarin (AMC) released from the peptide substrate LLVY-AMC (SEQ ID NO:8) as a result of the 20S proteasome activities. Proteasome samples were prepared according to the manufacturer's instructions. 25 μM of the thiazole antibiotics or proteasome inhibitors were pre-incubated with the proteasome samples for 15 minutes at room temperature before the proteasome substrate (LLVY-AMC) was added. Samples were incubated for 1 hour at 37° C. and the fluorescence derived from the cleaved AMC was detected and quantified using a 380/460 nm filter in a fluorometer (Spectra Max GeminiXS, Molecular Devices, Sunnyvale, Calif.).

As shown in FIG. 1C, cells treated with thiazole antibiotics Siomycin A or thiostrepton exhibited decreased amounts of free AMC indicating decreased proteolytic activity of the 20S proteasome, although the decrease was not as prominent as that by MG132 or lactacystin. This was the first time thiazole antibiotics have been shown to inhibit proteasome activity.

### Example 2

### Proteasome Inhibitors Inhibited FoxM1 Activity

The effects of proteasome inhibitors on FoxM1 transcriptional activity were tested by examining FoxM1-dependent luciferase activity in a luciferase assay. It was previously reported that thiazole antibiotics inhibited FoxM1-dependent transcriptional activity by measuring the reduction of luciferase activity in a U2OS-derived cell line stably expressing doxycycline-inducible FoxM1 and FoxM1-dependent luciferase (the U2OS-C3-Luc cell line). See U.S. Patent Application Publication No. 2008/0152618; Radhakrishnan et al. 2006; and Bhat et al., 2009. The same cell line was used in this experiment. U2OS-C3-Luc cells were treated with a combination of 1 μg/ml doxycycline and proteasome inhibitors at indicated concentrations for 24 hours, and the luciferase activity was measured. The luciferase activity was determined by using either the Luciferase Assay System (Promega, Madison, Wis.) or the Dual-Luciferase reporter assay system (Promega) according to the manufacturer's instructions.

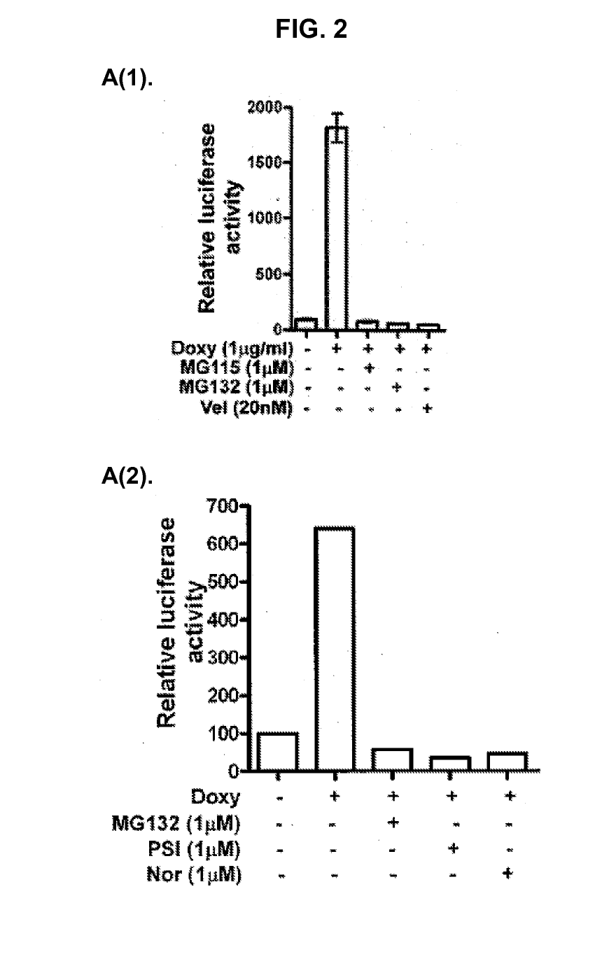

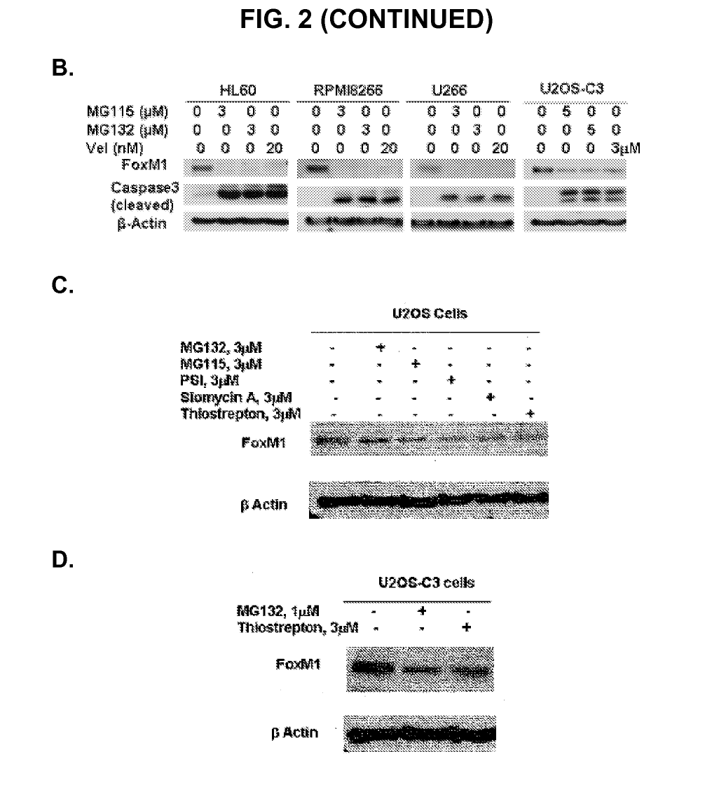

Similar to thiazole antibiotics, all proteasome inhibitors tested (MG115, MG132 and VELCADE® in FIG. 2A(1), and additionally PSI in FIG. 2A(2)) inhibited FoxM1 transcriptional activity as shown in the luciferase assay. The results shown in FIG. 2A (1) were normalized by the amount of proteins in the sample; and the results shown in FIG. 2A (2) were normalized using the Dual-luciferase reporter assay system (Promega).

The effects of proteasome inhibitors on FoxM1 protein expression were also examined. U266 and RPMI8226, both multiple myeloma cells, HL-60 leukemia cells and human U2OS osteosarcoma cells were treated with proteasome inhibitors MG115, MG132 and VELCADE® for 24 hours. Cell lysates were harvested and the FoxM1 protein levels were analyzed by immunoblot analysis. As shown in FIG. 2B, the levels of FoxM1 protein decreased in the presence of proteasome inhibitors. Similar to thiazole antibiotics, proteasome inhibitors decreased the levels of FoxM1 expression in a manner comparable to thiazole antibiotics. See FIGS. 2C and 2D. See also U.S. Patent Application Publication No. 2008/0152618.

Activation of caspase 3 has been used as an indicator of apoptosis. Caspase 3 exists as an inactive proenzyme, which is activated by proteolytic processing at conserved aspartic residues to produce subunits that dimerize to form the active enzyme. As shown in FIG. 2B, proteasome inhibitors decreased FoxM1 protein levels but increased the protein levels of the active, cleaved form of caspase 3 by immunoblot analysis.

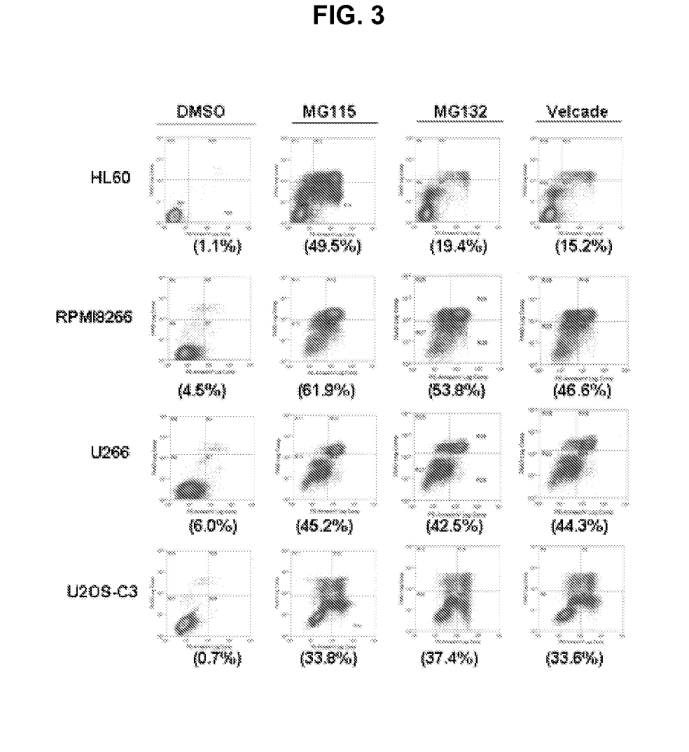

Proteasorne inhibitor-induced apoptosis was further quantified by using Annexin V-PE/7AAD staining. U266 and RPMI8226 multiple myeloma, HL-60 leukemia and U2OS osteosarcoma cells were cultured in the presence of proteasome inhibitors MG115, MG132 and VELCADE®. Following proteasome inhibitor treatment (or DMSO in the control), cells were stained with Annexin V-PE/7AAD (BD-Pharmingen, Franklin Lakes, N.J.) and then analyzed by Fluorescence-activated Cell Sorting (FACS). As shown in FIG. 3, not only did proteasome inhibitors decrease the protein levels of FoxM1, the proteasome inhibitors also induced apoptosis in the cells. The concentrations of proteasome inhibitors used in the experiments shown in FIG. 3 were the same as those in FIG. 2. The percentages of apoptotic cells were shown in the parentheses in FIG. 3. The induction of apoptosis correlated with the suppression of FoxM1 (see FIG. 3 and FIG. 2B).

### Example 3

### Over-Expression of FoxM1 Protected Apoptosis Induced by Proteasome Inhibitors

Over-expression of FoxM1 was often observed in a variety of tumors. See Pilarsky et al., 2004, Neoplasia 6(6):744-750; Chan et al., 2008, J Pathol 215(3):245-252. A U2OS-derived cell line stably expressing doxycycline-inducible FoxM1-GFP fusion protein (U2OS-C3) was used to examine the role of FoxM1 in apoptosis induced by proteasome inhibitors. Exogenous FoxM1 expression was induced by doxycycline and the following day the cells were treated with different concentrations of VELCADE® for 24 hours. As shown in FIG. 4, VELCADE®-induced active caspase 3 expression was reduced in cells that over-expressed FoxM1. In contrast, over-expression of FoxM1 did not protect against doxorubicin-induced caspase 3 expression, which suggested that FoxM1 specifically protected cells against proteasome inhibitor-induced apoptosis (FIG. 4). These results suggested that suppression of FoxM1 may be beneficial for cancer therapy using proteasome inhibitors.

### Example 4

### Combination of a Thiazole Antibiotic and a Proteasome Inhibitor Synergistically Induced Apoptosis in Tumor Cells

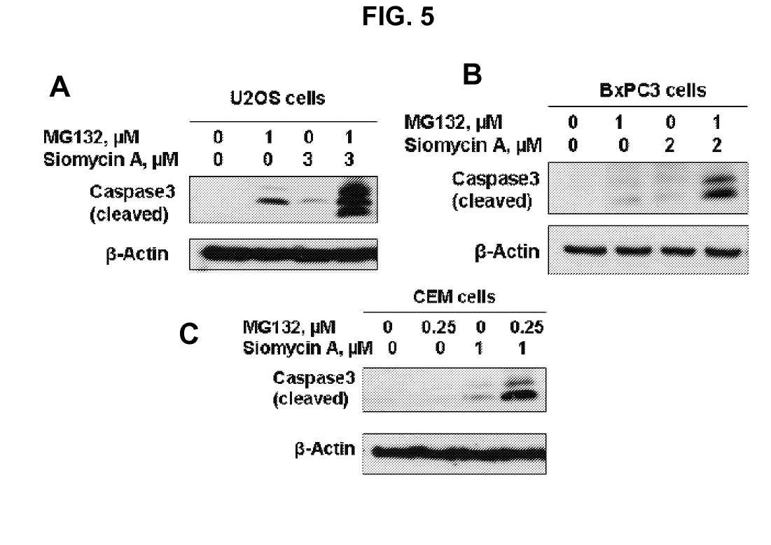

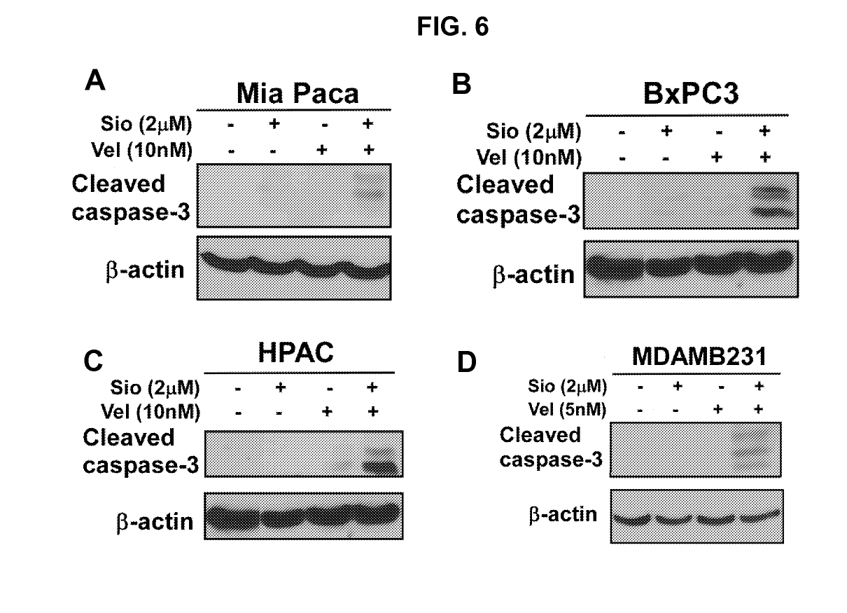

Currently approved anticancer therapy using the proteasome inhibitor VELCADE® has been hindered by the high toxicity of the compound. It was unexpected discovered in the instant application that less proteasome inhibitor was required to effectively induce apoptosis when a thiazole antibiotic was also used in conjunction with the proteasome inhibitor for treating tumor cells. As shown in FIG. 5, tumor cells treated with a combination of thiazole antibiotic Siomycin A and 1 μM MG132 induced expression of active caspase 3 to the levels comparable or higher as compared to the induction in cells treated with 3 μM MG132 alone (compare FIG. 2B with FIGS. 5A and 5B). MG132, even at a concentration as low as 0.25 μM, induced cleaved caspase 3 expression in the presence of 1 μM Siomycin A (FIG. 5C). Similar synergistic effects were seen in cells treated for 24 hours with 2 μM Siomycin A and 5 nM or 10 nM VELCADE® (FIG. 6). The synergistic effects of a thiazole antibiotic and a proteasome inhibitor on apoptosis were seen in different cell types: osteosarcoma cells (U2OS), human pancreatic cancer cells (BxPC3, Mia Paca, HPAC), lymphoblastic leukemia cells (CEM), human breast cancer cells (MDAMB231). When used alone, 5 μM Siomycin A was required to induce apoptosis in Mia Paca, BxPC3 and MDAMB231 cells, while 10-15 μM Siomycin A was required to induce apoptosis in HPAC cells (data not shown).

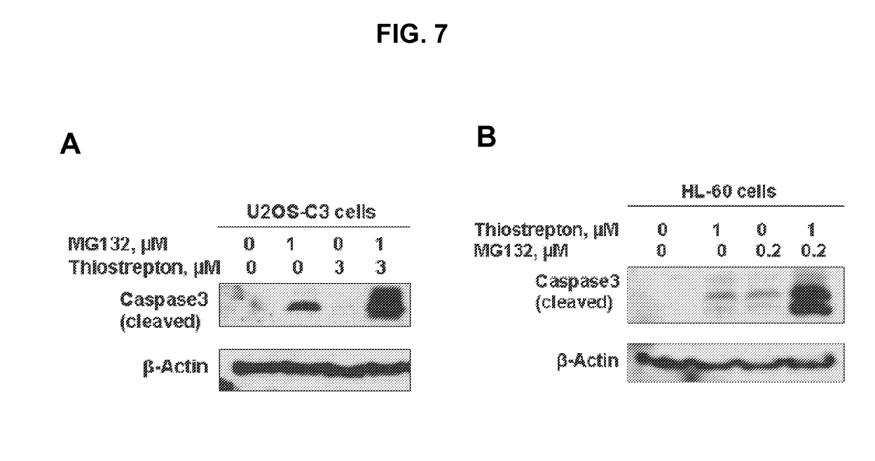

Similarly, thiostrepton and MG132 synergistically induced the active form of caspase 3 expression in different tumor cells. As shown in FIG. 7, induction of caspase 3 expression by 0.2 μM MG132 was greatly enhanced by the presence of 1 μM of thiostrepton (see FIG. 7B). The synergistic effects were seen in different cell types: osteosarcoma cells (U2OS-C3, uninduced by doxycycline), and leukemia cells (HL-60). The synergistic effects were also seen in neuroblastoma cells (IMR32 cells, data not shown).

Separate experiments were conducted in prostate cancer cells to examine the synergistic effects of thiosstrepton and VELCADE® on apoptosis. Prostate cancer DU145 and PC3 cells were treated with DMSO (control), a single agent (1.5 μM thiostrepton or 7.5 nM VELCADE®) or a combination thereof for 48 hrs, and the levels of cleaved caspase-3 were determined by immunoblot analysis. The concentrations used in the combination, 1.5 μM for thiostrepton and 7.5 nM for VELCADE®, were much lower than the minimal concentrations required for induction of caspase 3 expression by each compound individually, 3 μM for thiostrepton alone and 50 nM for VELCADE® alone, as previously determined in prostate cancer cells (data not shown).

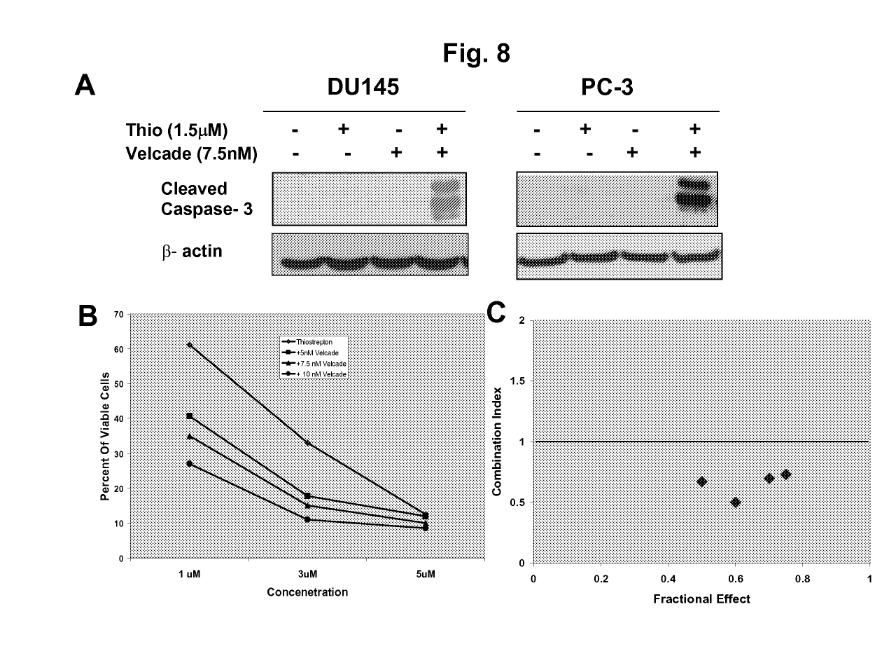

As shown in FIG. 8, thiostrepton and VELCADE® synergistically induced caspase 3 expression in prostate cancer cells at concentrations much lower than what would be required if each compound was used alone (FIG. 8A). The synergistic effects on cell viability were determined on the basis of the dose-response curves obtained using standard MTT assay according to the manufacturer's instructions (the kit can be obtained from for example, Biotium, Inc. Hayward, Calif.) (FIG. 8B). In short, the compound 3-(4,5-Dimethylthiazol-2-yl)-2,5-diphenyltetrazolium bromide (MTT) was added to the cells. MTT was reduced by cellular dehydrogenase into formazan, which is soluble in tissue culture medium. Measuring formazan absorbance at a wavelength of 492 nm reflected levels of dehydrogenase enzyme activity found in metabolically-active cells. Since the production of formazan is proportional to the number of living cells, the intensity of the produced color is a good indication of the viability of the cells. The cell viability in each experiment as a percentage to the control was plotted against the concentrations of thiostrepton. As shown in FIG. 8B, thiostrepton and VELCADE® synergistically reduced viability of prostate cancer cells when the cells were treated with the two compounds combined.

Combination index (CI) values were calculated for different dose-effect levels. The CI values for a combination of 0.8 μM of thiostrepton with 7.5 nM of VELCADE®, and 1.5 μM of thiostrepton with 7.5 nM of VELCADE® were 0.65 and 0.74, respectively (data not shown). Similarly a CI value of 0.5 was demonstrated by a combined treatment of 1 μM of thiostrepton with 10 nM of VELCADE®. The CI values of <1 indicated synergy, a value of 1 would indicate additive effects and a value of >1 would indicate antagonism.

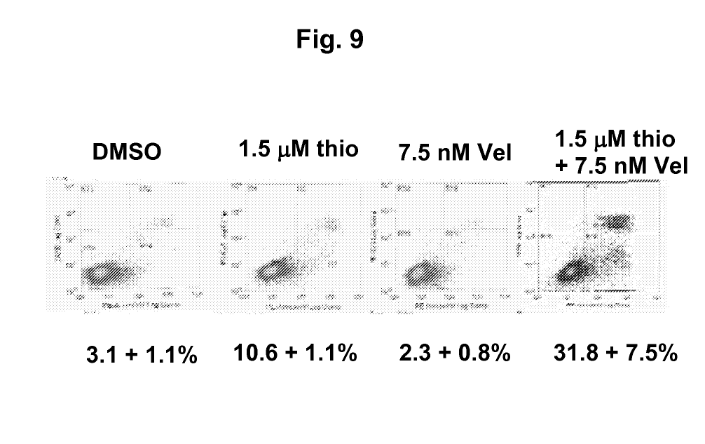

Apoptosis in prostate cancer cells treated with 1.5 mM thiostrepton or 7.5 nM VELCADE®, separately or in combination was confirmed by FACS analysis. After drug treatment (or DMSO in control), cells were stained with Annexin V-PE/7AAD for FACS analysis (FIG. 9, percentage of apoptotic cells was shown under each panel). As shown in FIG. 9, combination of thiostrepton and VELCADE® greatly increased the percentage of apoptotic cells.

It should be understood that the foregoing disclosure emphasizes certain specific embodiments of the invention and that all modifications or alternatives equivalent thereto are within the spirit and scope of the invention as set forth in the appended claims.

1-38. (canceled)

39. A pharmaceutical composition for inducing apoptosis in a tumor cell, comprising a proteasome inhibitor and a thiazole antibiotic, and at least one excipient, diluent or carrier, wherein the combination of the proteasome inhibitor and the thiazole antibiotic is effective in inducing apoptosis in the tumor cell.

40. The pharmaceutical composition of claim 39, wherein the proteasome inhibitor is MG132, MG115, Velcade, lactacystin, or PSI.

41. The pharmaceutical composition of claim 39, wherein the thiazole antibiotic is Siomycin A or thiostrepton.

42. The pharmaceutical composition of claim 39 comprising a suboptimal amount of the proteasome inhibitor and a suboptimal amount of the thiazole antibiotic, wherein the combination of the proteasome inhibitor and thiazole antibiotic induces apoptosis in the tumor cell.

43. The pharmaceutical composition of claim 42, wherein the proteasome inhibitor is present in the suboptimal amount from about 2 μg/kg to about 400 μg/kg.

44. The pharmaceutical composition of claim 42, wherein the thiazole antibiotic is present in the suboptimal amount from about 800 μg/kg to about 5 mg/kg.

45. A method for inducing apoptosis in a tumor cell comprising the step of contacting the tumor cell with the pharmaceutical composition according to claim 39 comprising a proteasome inhibitor and a thiazole antibiotic, wherein the combination of the proteasome inhibitor and the thiazole antibiotic is effective in inducing apoptosis in the tumor cell.

46. The method of claim 45, wherein the proteasome inhibitor is MG132, MG115, Velcade, lactacystin, or PSI.

47. The method of claim 46, wherein the proteasome inhibitor is Velcade.

48. The method of claim 45, wherein the thiazole antibiotic is Siomycin A, thiostrepton, sporangiomycin, nosiheptide, multhiomycin, micrococcin or thiocillin.

49. The method of claim 48, wherein the thiazole antibiotic is Siomycin A or thiostrepton.

50. The method of claim 49, wherein the proteasome inhibitor is Velcade and the thiazole antibiotic is thiostrepton.

51. The method of claim 45, wherein the proteasome inhibitor is present in a suboptimal amount in the pharmaceutical composition and the thiazole antibiotic is present in a suboptimal amount in the pharmaceutical composition, and wherein the combination of the proteasome inhibitor and the thiazole antibiotic induces apoptosis in the tumor cell.

52. The method of claim 51, wherein the suboptimal amount of the proteasome inhibitor is from about 2 μg/kg to about 400 μg/kg.

53. The method of claim 51, wherein the suboptimal amount of the thiazole antibiotic is from about 800 μg/kg to about 5 mg/kg.

54. A pharmaceutical composition for inducing apoptosis in a tumor cell, comprising a proteasome inhibitor and an agent that reduces FoxM1 activity, and at least one excipient, diluent or carrier, wherein the combination of the proteasome inhibitor and the agent that reduces FoxM1 activity is effective in inducing apoptosis in the tumor cell.

55. The pharmaceutical composition of claim 54, wherein the agent is a thiazole antibiotic, a FoxM1 siRNA, or a p19ARF peptide.

56. The pharmaceutical composition of claim 55, wherein the agent is a thiazole antibiotic.

57. The pharmaceutical composition of claim 56, wherein the thiazole antibiotic is Siomycin A or thiostrepton.

58. The pharmaceutical composition of claim 54, wherein the proteasome inhibitor is MG132, MG115, Velcade, lactacystin, or PSI.

59. A method for inducing apoptosis in a tumor cell that expresses FoxM1 protein, comprising the step of contacting the tumor cell with the pharmaceutical composition according to claim 54 comprising a proteasome inhibitor and at least one agent that reduces FoxM1 activity.

60. The method of claim 59, wherein the agent is a thiazole antibiotic, a FoxM1 siRNA, or a p19ARF peptide.

61. The method of claim 60, wherein the agent is a thiazole antibiotic.

62. The method of claim 61, wherein the thiazole antibiotic is Siomycin A or thiostrepton.

63. The method of claim 59, wherein the proteasome inhibitor is MG132, MG115, Velcade, lactacystin, or PSI.

64. The method of claim 63, wherein the proteasome inhibitor is Velcade.

65. A method for inhibiting FoxM1 activity in a tumor cell, the method comprising the step of contacting the cell with a proteasome inhibitor.

66. The method of claim 65, wherein the proteasome inhibitor is MG132, MG115, Velcade, lactacystin, or PSI.

67. The method of claim 66, wherein the proteasome inhibitor is Velcade.

68. The method of 65, further comprising contacting the tumor cell with a thiazole antibiotic.

69. The method of claim 68, wherein the thiazole antibiotic is Siomycin A, thiostrepton, sporangiomycin, nosiheptide, multhiomycin, micrococcin or thiocillin.

70. The method of claim 69, wherein the thiazole antibiotic is Siomycin A or thiostrepton.

71. A method for inhibiting proteasome activity in a cell, the method comprising the step of contacting the cell with a thiazole antibiotic.

72. The method of claim 71 wherein the thiazole antibiotic is Siomycin A, thiostrepton, sporangiomycin, nosiheptide, multhiomycin, micrococcin or thiocillin.

73. The method of claim 72, wherein the thiazole antibiotic is Siomycin A or thiostrepton.

74. A method for identifying a compound that has proteasome inhibitory activity in a cell by determining the reduction of FoxM1 activity in the cell by the compound, wherein the cell expresses FoxM1, the method comprising the steps of contacting the cell with the compound, and assaying for FoxM1 activity in the cell.

75. The method of claim 74, wherein the compound is a thiazole antibiotic compound.

76. A method for identifying a thiazole antibiotic that inhibits proteasome activity in a cell, comprising the steps of contacting the cell with said thiazole antibiotic and detecting reduced proteasome activity in the cell.


SEQUENCE LISTING (9 sequences)

SEQ ID NO: 1 - RNA, 21 nt, Artificial Sequence
  Synthetic siRNA
caacaggagucuaaucaaguu

SEQ ID NO: 2 - RNA, 21 nt, Artificial Sequence
  Synthetic siRNA
ggaccacuuucccuacuuuuu

SEQ ID NO: 3 - RNA, 21 nt, Artificial Sequence
  Synthetic siRNA
guagugggcccaacaaauuuu

SEQ ID NO: 4 - RNA, 21 nt, Artificial Sequence
  Synthetic siRNA
gcugggaucaagauuauuauu

SEQ ID NO: 5 - protein, 28 aa, Artificial Sequence
  Synthetic
XXXXXXXXXKFVRSRRPRTASCALAFVN

SEQ ID NO: 6 - protein, 19 aa, Artificial Sequence
  Synthetic
KFVRSRRPRTASCALAFVN

SEQ ID NO: 7 - protein, 30 aa, Artificial Sequence
  Synthetic
KFVRSRRPRTASCALAFVNMLLRLERILRR

SEQ ID NO: 8 - protein, 4 aa, Artificial Sequence
  Synthetic
LLVY

SEQ ID NO: 9 - protein, 4 aa, Artificial
  Synthetic
IEAL